(19) United States
(12) Patent Application Publication
(10) Pub. No.: US 2018/0035908 A1
(43) Pub. Date: Feb. 8, 2018

(54) ELECTROCARDIOGRAM MEASURING DEVICE

(71) Applicant: EKG TECHNOLOGY LAB, INC., Funabashi-shi (JP)

(72) Inventor: DAMING WEI, Funabashi-shi (JP)

(21) Appl. No.: 15/667,179

(22) Filed: Aug. 2, 2017

(30) Foreign Application Priority Data
Aug. 5, 2016 (JP) 2016-154434

(51) Int. Cl. A61B 5/0408; A61B 5/00

(52) U.S. Cl. CPC A61B 5/04085; A61B 5/6804; A61B 5/6891

(57) ABSTRACT

An electrocardiogram measuring device includes: chest measuring electrodes 113 that acquire electrocardiographic signals of a chest; a T-shirt 100 having an attachment holes of the chest measuring electrodes opened; a chair 200 on which limb measuring electrodes 212 and 214 that acquire electrocardiographic signals of limbs are arranged; and an electrocardiogram generating unit 310 that generates an electrocardiogram of a subject from the electrocardiographic signals of the chest and the limbs respectively acquired by the chest measuring electrodes attached to a body surface of the subject through the attachment holes of the T-shirt 100 and the limb measuring electrodes with which both hands and feet of the subject are brought into contact.

### CROSS-REFERENCE TO RELATED APPLICATION

The present application claims priority under 35 U.S.C. § 119 to Japanese Patent Application No. 2016-154434, filed Aug. 5, 2016. The contents of this application are incorporated herein by reference in their entirety.

### BACKGROUND

### 1. Technical Field

The present invention relates to an electrocardiogram measuring device with which a subject can take an electrocardiogram by him/herself.

### 2. Description of Related Arts

Generally, as an electrocardiogram, a 12-lead electrocardiogram is used as an industry standard. When the 12-lead electrocardiogram is taken, a subject lies on a simple bed, and a measurer attaches electrodes for limb lead measurement to four positions, both wrists and both ankles, and electrodes for chest lead measurement to predetermined six positions of a chest of the subject, respectively.

In this way, since it is necessary that the measurer attaches the electrodes to the predetermined 10 positions of the subject's body for taking the 12-lead electrocardiogram, measurement of the 12-lead electrocardiogram can only be performed at a medical institution such as a hospital. Thus, the measuring operation becomes restrictive, and the subject is forced to have some conscious tension. For this reason, it is difficult to measure the 12-lead electrocardiogram at home.

In recent years, as the aging progresses, there is a desire to make it possible to measure the 12-lead electrocardiogram at home. However, a general subject generally does not know exact attachment positions of the electrodes, and thus depends only on measurement at a hospital. An electrocardiograph that the subject him/herself can attach a plurality of electrodes is described in Japanese Patent Publication No. 2012-49195. However, even if this electrocardiograph is applied, it is difficult for the subject to make it possible to measure the 12-lead electrocardiogram by him/herself.

### SUMMARY

The present invention is made to solve the problems of the conventional electrocardiograph, and has an object to provide an electrocardiogram measuring device with which a subject can take an electrocardiogram by him/herself.

An electrocardiogram measuring device according to the present invention for achieving the above object, includes: chest measuring electrodes that acquire electrocardiographic signals of a chest; a garment having attachment holes of the chest measuring electrodes opened; a chair on which limb measuring electrodes that acquire electrocardiographic signals of limbs are arranged; and an electrocardiogram generating unit that generates an electrocardiogram of a subject from the electrocardiographic signals of the chest and the limbs respectively acquired by the chest measuring electrodes attached to a body surface of the subject through the attachment holes of the garment and the limb measuring electrodes with which both hands and feet of the subject are brought into contact.

According to the present invention, since the subject him/herself can attach the chest measuring electrodes to optimal positions and the limb lead electrodes are arranged on the chair, the subject can unconstrainedly and unconsciously measure the electrocardiogram by him/herself. Thus, measurement of the electrocardiogram can be easily performed at home other than a medical institution, for example.

### BRIEF DESCRIPTION OF THE DRAWINGS

FIG. 1 shows a configuration of a garment of an electrocardiogram measuring device according to the present embodiment.

FIG. 2 is a diagram in which chest measuring electrodes of the electrocardiogram measuring device according to the present embodiment is attached to a subject.

FIG. 3 shows a configuration of the chest measuring electrode.

FIG. 4 shows a use mode of a chair of the electrocardiogram measuring device according to the present embodiment.

FIG. 5 shows a configuration of the chair shown in FIG. 4.

FIG. 6 shows a configuration of a conductive sheet used for the chair in FIGS. 4 and 5.

FIG. 7 is a block diagram of a control system of the electrocardiogram measuring device according to the present embodiment.

FIG. 8 is a flow chart showing a measurement procedure of an electrocardiogram using the electrocardiogram measuring device according to the present embodiment.

### DETAILED DESCRIPTION

Next, an electrocardiogram measuring device according to the present embodiment will be described in detail with reference to the drawings.

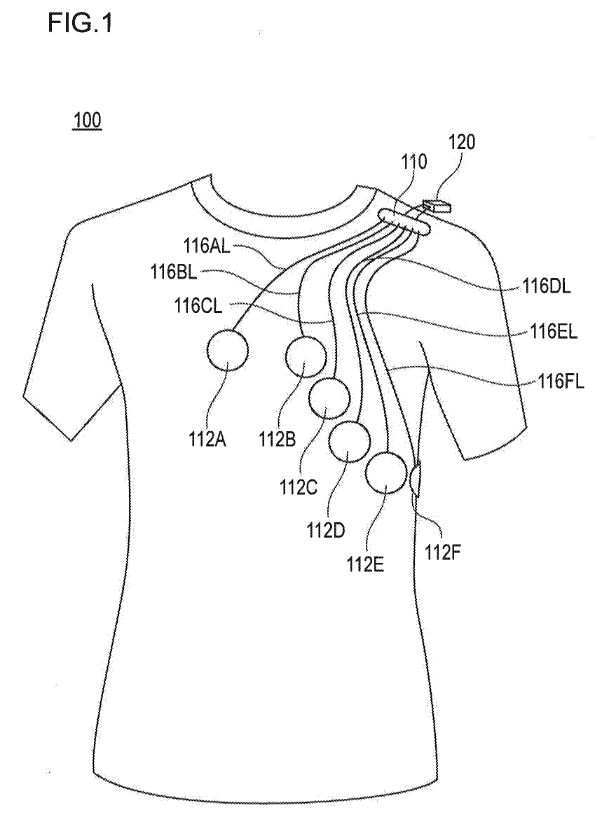

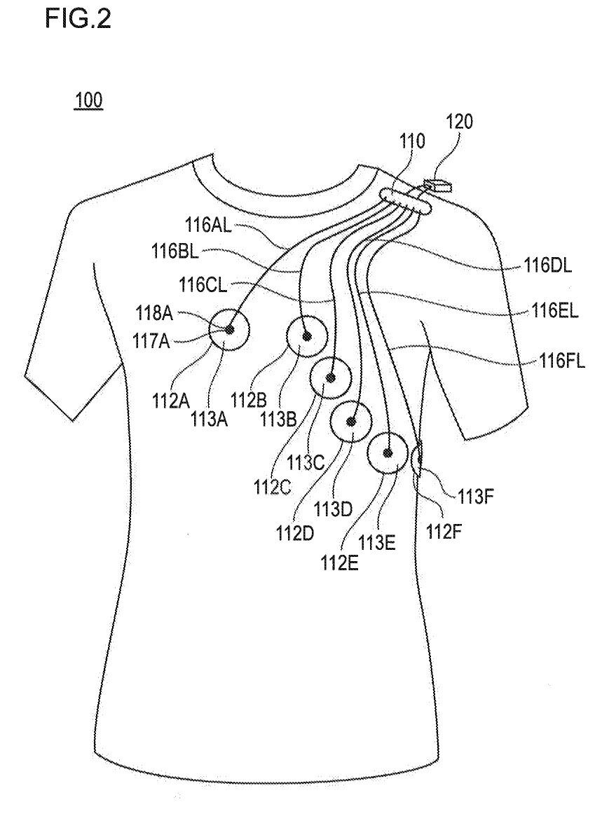

FIG. 1 shows a configuration of a garment of an electrocardiogram measuring device according to the present embodiment, and FIG. 2 is a diagram in which chest measuring electrodes of the electrocardiogram measuring device according to the present embodiment is attached to a subject. In the present embodiment, a T-shirt 100 is used as a garment. However, in addition to the T-shirt, a Y-shirt, a polo shirt and the like can also be used as a garment. As shown in FIG. 2, in the present embodiment, in order to attach six chest measuring electrodes 113A to 113F that acquire electrocardiographic signals of a chest, a general T-shirt has attachment holes 112A to 112F (white portions in the figure) opened, as shown in FIG. 1. An opening diameter of the attachment holes 112A to 112F is equal to or larger than a size of the chest measuring electrodes 113A to 113F.

The attachment holes 112A to 112F opened in the T-shirt 100 are arranged at predetermined positions of the subject by measurements, for acquiring optimal electrocardiographic signals of the subject. Optimal positions of the respective chest measuring electrodes 113A to 113F are different depending on the subject. Accordingly, the positions of the attachment holes 112A to 112E are determined so that the chest measuring electrodes 113A to 113F are attached to the positions optimal for the subject. The attachment holes 112A to 112F serve as guides when the subject him/herself attaches the chest measuring electrodes 113A to 113F. It is to be noted that, in the present embodiment, six attachment holes 112A to 112F opened in the general T-shirt are illustrated, but attachment holes other than six attachment holes (for example, 1 to 5, 7 or more attachment holes) may be opened in the T-shirt to correspond to an electrocardiogram other than a 12-lead electrocardiogram.

The T-shirt 100 has a garment pad part 110 that bundles lead wires 116AL to 116FL (corresponding to chest measuring electrode lead wires) connected to the chest measuring electrodes 113A to 113F on a shoulder part of the T-shirt 100. A garment pad part connector 120 to which the bundled lead wires 116AL to 116FL are connected is attached to the garment pad part 110. Since the lead wires 116AL to 116FL are bundled by the garment pad part 110, they are loosened when not attached to the chest measuring electrodes 113A to 113F. To prevent the above situation, the garment may be provided with a stopper part that restrains each lead wire 116AL to 116FL.

The chest measuring electrodes 113A to 113F have male connecting parts 117A for connecting the lead wires 116AL to 116FL, and the lead wires 116AL to 116FL have female connecting parts 118A for connecting to the male connecting parts 117A.

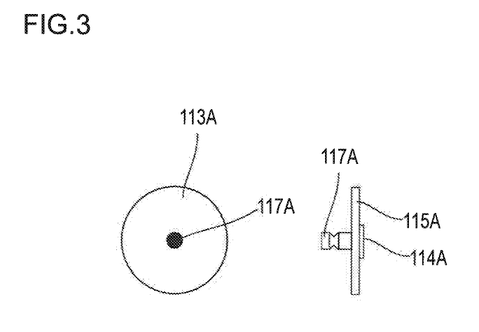

FIG. 3 shows a configuration of the chest measuring electrode 113A. As shown, an electrode part 114A that directly contacts a body surface of the subject, and an adhesive part 115A for bringing the electrode part 114A into close contact with the body surface are provided on a back surface of the chest measuring electrode 113A. The electrode part 114A penetrates through the adhesive part 115A to be connected to the male connecting part 117A. The male connecting part 117A of the chest measuring electrode 113A engages with the female connecting part 118A provided on a tip end of the lead wire 116AL (see FIG. 2).

Since the T-shirt 100, the chest measuring electrodes 113A to 113F, and the lead wires 116AL to 116FL are configured as described above, when measuring the 12-lead electrocardiogram, the subject can complete measurement preparation of the chest lead by simply wearing the T-shirt 100, attaching the chest measuring electrodes 113A to 113F into the attachment holes 112A to 112F, and attaching the lead wires 116AL to 116FL to the chest measuring electrodes 113A to 113F.

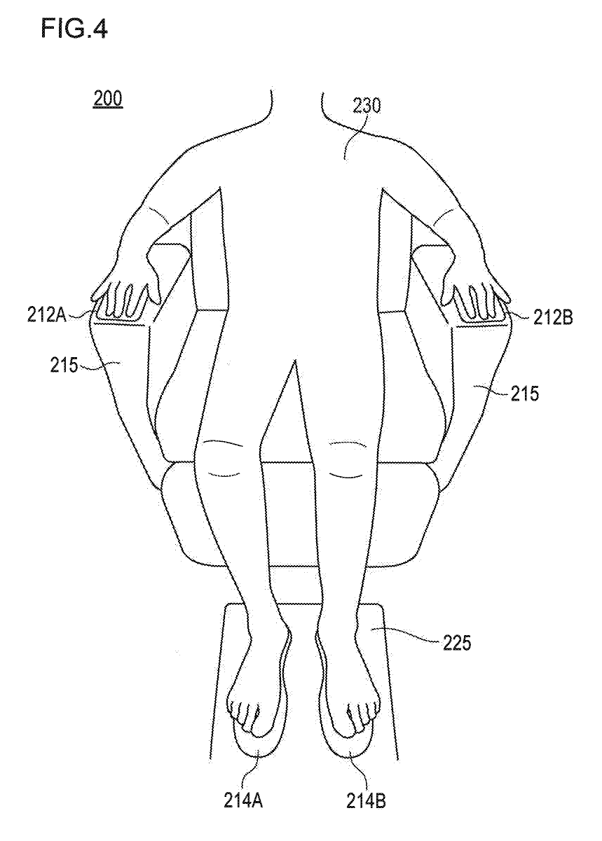

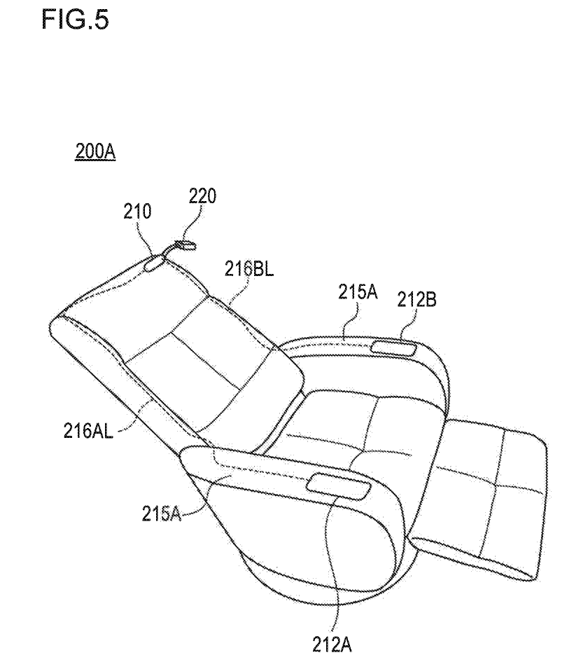

FIG. 4 shows a use mode of a chair of the electrocardiogram measuring device according to the present embodiment. Furthermore, FIG. 5 shows a configuration of the chair shown in FIG. 4. As shown in FIGS. 4 and 5, a chair 200 has armrest parts 215 on which both arms of a subject 230 are placed, and a footrest part 225 on which both feet of the subject 230 are placed. Limb measuring electrodes 212A and 212B that acquire electrocardiographic signals from both hands of the subject 230 are arranged on the armrest parts 215. Limb measuring electrodes 214A and 214B that acquire electrocardiographic signals from both feet of the subject 230 are arranged on the footrest part 225. Note that when the electrocardiogram of the limbs are taken with the posture as shown in FIG. 4, it is sufficient to place the foot of the subject 230 on the footrest part 225 as in FIG. 4. However, when the electrocardiogram of the limbs are taken with the reclined posture as in FIG. 5, the footrest part 225 cannot be used, so that a clip electrodes are attached to ankles of the subject 230.

The chair 200 has a chair pad part 210 that bundles lead wires (216AL and 216BL shown in FIG. 5, corresponding to limb measuring electrode lead wires) that are connected to the limb measuring electrodes 212A, 212B, 214A, and 214B (see FIGS. 4 and 5), and a chair pad part connector 220 to which the bundled lead wires are connected is attached to the chair pad part 210 (see FIG. 5).

The lead wire 216AL connects the limb measuring electrodes 212A and 214A with the chair pad part 210. The lead wire 216BL connects the limb measuring electrodes 212B and 214B with the chair pad part 210. The lead wires 216AL and 216BL are routed within a structural material of a chair 200A from the respective limb measuring electrodes 212A, 214A, 212B and 214B to the chair pad part 210.

Since the chair 200 has a reclining function, the subject can relax during measurement.

The limb measuring electrodes 212A and 212B of the armrest parts 215 are flat, and have an area to the extent that the entire palm of the subject 230 can be placed. The limb measuring electrodes 212A and 212B of the armrest parts 215 have surfaces on which conductive sheets (not shown, corresponding to armrest part conductive sheets) are mounted, and acquire electrocardiographic signals by putting of the palm of the subject 230 as shown in FIG. 4. Furthermore, the limb measuring electrodes 214A and 214B of the footrest part 225 are flat, and have an area to the extent that the entire foot sole of the subject 230 can be placed. The limb measuring electrodes 214A and 214B of the footrest part 225 have surfaces on which conductive sheets (not shown, corresponding to footrest part conductive sheets) are mounted, and acquire electrocardiographic signals by putting of the foot sole of the 230 as shown in FIG. 4.

As shown in FIG. 5, the chair 200 has the chair pad part 210 that bundles the lead wires connected to the limb measuring electrodes 212A, 212B, 214A, and 214B on a shoulder part of a backrest part of the chair 200. The chair pad part connector 220 to which the bundled lead wires are connected is attached to the chair pad part 210.

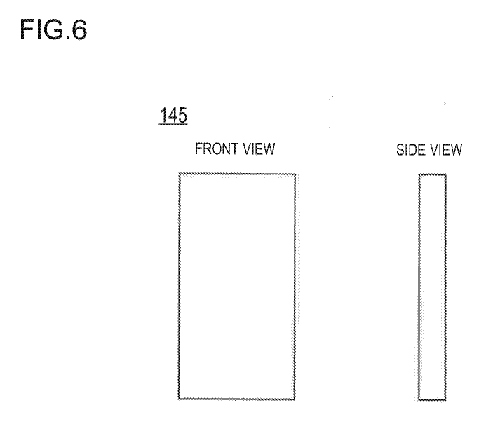

FIG. 6 shows a configuration of a conductive sheet used for the chair in FIGS. 4 and 5. The conductive sheets is mounted on the limb measuring electrodes 212A, 212B, 214A, and 214B.

A conductive sheet 145 has a quadrangular shape when viewed from the plane according to a shape of the limb measuring electrodes 212A, 212B, 214A, and 214B. The conductive sheet 145 is a gel-like sheet having a thickness of about several millimeters when viewed from the side face. When the palm or the foot sole is put on the conductive sheet 145, the conductive sheet 145 deforms and adheres to conform to a shape of the surface of the palm or the foot sole. The limb electrocardiographic signals of the subject is transmitted to the limb measuring electrodes 212A, 212B, 214A, and 214B via the conductive sheets 145.

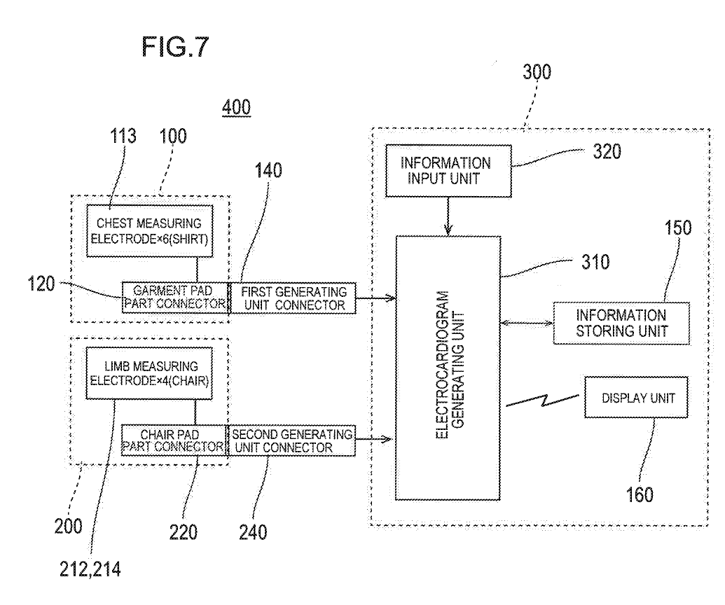

FIG. 7 is a block diagram of a control system of the electrocardiogram measuring device according to the present embodiment. An electrocardiogram measuring device 400 according to the present embodiment includes the T-shirt 100, the chair 200, and a control unit 300.

As shown in FIG. 2, the T-shirt 100 includes six chest measuring electrodes 113 (the general name of the chest measuring electrodes 113A to 113F in FIG. 1), and the garment pad part connector 120 connected to these chest measuring electrodes 113.

As shown in FIGS. 4 and 5, the chair 200 includes four limb measuring electrodes 212 and 214 (the general name of the limb measuring electrodes 212A and 212B and the limb measuring electrodes 214A and 214B in FIG. 2), and the chair pad part connector 220 connected to these limb measuring electrodes 212 and 214.

The control unit 300 includes an electrocardiogram generating unit 310, an information input unit 320, an information storing unit 150, and a display unit 160. A first generating unit connector 140 for collectively connecting garment connecting electric wires connected to the electrocardiogram generating unit 310 to the garment pad part connector 120, and a second generating unit connector 240 for collectively connecting chair connecting electric wires connected to the electrocardiogram generating unit 310 to the chair pad part connector 220 are attached to the electrocardiogram generating unit 310.

The electrocardiogram generating unit 310 generates an electrocardiogram of the subject from the electrocardiographic signals of the chest and the limbs respectively acquired by the chest measuring electrodes 113 attached to the body surface of the subject and the limb measuring electrodes 212 and 214 with which both hands and feet of the subject are brought into contact. A conventional generating method of the 12-lead electrocardiogram is used for generation of the electrocardiogram.

The information input unit 320 inputs subject information such as sex and age of the subject. The display unit 160 displays the electrocardiogram generated by the electrocardiogram generating unit 310. The electrocardiogram generating unit 310 and the display unit 160 are wirelessly connected to each other. The information storing unit 150 stores the subject information input by the information input unit 320 and the electrocardiogram generated by the electrocardiogram generating unit 310.

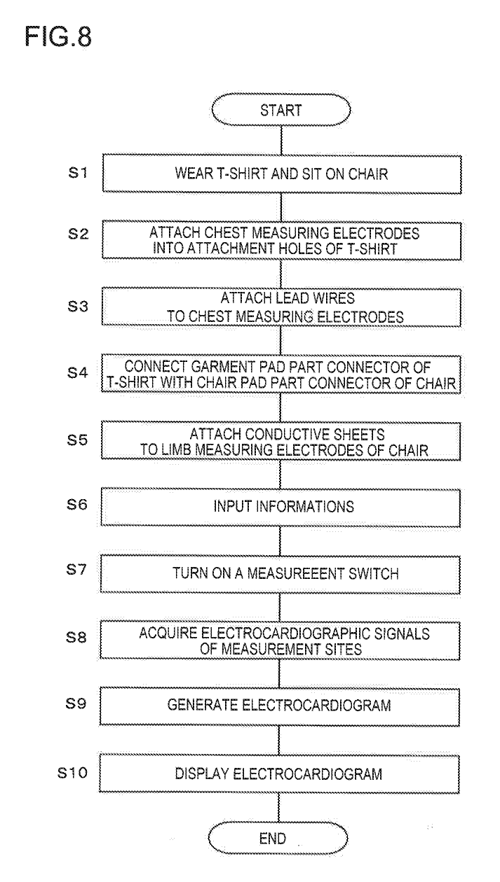

FIG. 8 is a flow chart showing a measurement procedure of an electrocardiogram using the electrocardiogram measuring device according to the present embodiment. The operation of this flow chart will be described in detail with reference to FIGS. 1 to 7. In this flow chart, the procedure from step S1 to step S7 is performed by the subject. Next, the procedure from step S8 to step S10 is performed by the electrocardiogram generating unit 310 shown in FIG. 7.

When the subject tries to take an electrocardiogram, the subject first wears the T-shirt 100 as shown in FIG. 1, and sits on the chair 200 or 200A as shown in FIG. 4 or FIG. 5 (S1).

The subject attaches the chest measuring electrodes 113A to 113F into the attachment holes 112A to 112F of the T-shirt 100. Since the positions of the attachment holes 112A to 112F are the predetermined positions of the subject, the subject can take the optimal electrocardiographic signals of the chest by simply attaching the chest measuring electrodes 113A to 113F into the attachment holes 112A to 112F (S2).

The subject connects the female connecting parts 118A of the lead wires 116AL to 116FL to the male connecting parts 117A of the chest measuring electrodes 113A to 113F (see FIGS. 2 and 3). As a result, the lead wires 116AL to 116FL are attached to the chest measuring electrodes 113A to 113F (S3).

Next, the subject couples the garment pad part connector 120 of the T-shirt 100 shown in FIGS. 1 and 2 with the first generating unit connector 140 shown in FIG. 7, and connects the chest measuring electrodes 113A to 113F of the T-shirt 100 to the electrocardiogram generating unit 310. Furthermore, the subject couples the chair pad part connector 220 of the chairs 200 and 200A shown in FIGS. 4 and 5 with the second generating unit connector 240 shown in FIG. 7, and connects the limb measuring electrodes 212A and 212B, and the limb measuring electrodes 214A and 214B to the electrocardiogram generating unit 310 (S4).

Next, the subject attaches the conductive sheets 145 shown in FIG. 6 to the limb measuring electrodes 212A and 212B arranged on the armrest parts 215A, and to the limb measuring electrodes 214A and 214B arranged on the footrest part 225 of the chairs 200 and 200A shown in FIG. 4 or FIG. 5 (S5).

Next, the subject inputs the subject information such as sex and age of the subject from the information input unit 320. The subject information is input so that an accurate electrocardiogram according to the sex and age can be generated (S6). The subject turns on a measurement switch (not shown) of the electrocardiogram measuring device 400 (S7).

The subject puts the palms on the conductive sheets 145 of the limb measuring electrodes 212A and 212B, puts the foot soles on the conductive sheets 145 of the limb measuring electrodes 214A and 214B, and performs measurement of the electrocardiogram in a relaxed state.

The electrocardiogram generating unit 310 acquires the electrocardiographic signals of measurement sites of the subject from the chest measuring electrodes 113 and the limb measuring electrodes 212 and 214 (S8). The electrocardiogram generating unit 310 creates the electrocardiogram using the acquired electrocardiographic signals (S9).

Based on cardiac potentials detected by 10 electrodes attached to the subject, the electrocardiogram generating unit 310 calculates limb 6-lead waveforms (I, II, III, aVR, aVL, and aVF) of a standard 12 lead and chest 6-lead waveforms (V1, V2, V3, V4, V5, and V6) of the standard 12 lead.

Generally, a relationship between the lead waveforms for obtaining a standard 12-lead electrocardiogram and the cardiac potentials at the measurement sites is as follows.

| | | |
|---|---|---|
| I | lead: | vL − vR |
| II | lead: | vF − vR |
| III | lead: | vF − vL |
| aVR | lead: | vR − (vL + vF)/2 |
| aVL | lead: | vL − (vR + vF)/2 |
| aVF | lead: | vF − (vL + vR)/2 |
| V1 | lead: | v1 − (vR + vL + vF)/3 |
| V2 | lead: | v2 − (vR + vL + vF)/3 |
| V3 | lead: | v3 − (vR + vL + vF)/3 |
| V4 | lead: | v4 − (vR + vL + vF)/3 |
| V5 | lead: | v5 − (vR + vL + vF)/3 |
| V6 | lead: | v6 − (vR + vL + vF)/3 |

Where, v is a potential detected at an each electrode attaching position.

Then, the display unit 160 displays the electrocardiogram generated by the electrocardiogram generating unit 310 (S10).

As described above, according to the electrocardiogram measuring device of the present embodiment, the electrocardiographic signals of the chest and the electrocardiographic signals of the limbs can be acquired respectively by the chest measuring electrodes attached into the attachment holes of the T-shirt and the limb measuring electrodes attached to the chair. Accordingly, the subject can acquire the electrocardiogram by simply wearing the T-shirt, attaching the chest measuring electrodes, and sitting on the chair, and thus the subject can take the electrocardiogram by him/herself.

Accordingly, the subject does not need to go to a hospital for taking the electrocardiogram, and can comfortably perform measurement at his/her home.

What is claimed is:

1. An electrocardiogram measuring device comprises:
chest measuring electrodes that acquire electrocardiographic signals of a chest;
a garment having attachment holes of the chest measuring electrodes opened;
a chair on which limb measuring electrodes that acquire electrocardiographic signals of limbs are arranged; and
an electrocardiogram generating unit that generates an electrocardiogram of a subject from the electrocardiographic signals of the chest and the limbs respectively acquired by the chest measuring electrodes attached to a body surface of the subject through the attachment holes of the garment and the limb measuring electrodes with which both hands and feet of the subject are brought into contact.

2. The electrocardiogram measuring device according to claim 1, wherein the garment has a garment pad part that bundles chest measuring electrode lead wires connected to the chest measuring electrodes, and
a garment pad part connector to which the chest measuring electrode lead wires are connected is attached to the garment pad part to collectively connect the bundled chest measuring electrode lead wires to the electrocardiogram generating unit.

3. The electrocardiogram measuring device according to claim 2, wherein a first generating unit connector to which garment connecting electric wires are connected is attached to the electrocardiogram generating unit to connect the garment connecting electric wires connected to the electrocardiogram generating unit to the garment pad part connector.

4. The electrocardiogram measuring device according to claim 2, wherein the chest measuring electrodes have male connecting parts for connecting the chest measuring electrode lead wires, and the chest measuring electrode lead wires have female connecting parts for connecting to the male connecting parts.

5. The electrocardiogram measuring device according to claim 1, wherein the attachment holes opened in the garment are arranged at predetermined positions of the subject to acquire optimal electrocardiographic signals of the subject.

6. The electrocardiogram measuring device according to claim 1, wherein the chair has armrest parts on which both arms of the subject are placed, and footrest parts on which both feet of the subject are placed,
the limb measuring electrodes are arranged on the armrest parts and the footrest parts,
the chair has a chair pad part that bundles limb measuring electrode lead wires connected to the limb measuring electrodes, and
a chair pad part connector to which the limb measuring electrode lead wires are connected is attached to the chair pad part to collectively connect the bundled limb measuring electrode lead wires to the electrocardiogram generating unit.

7. The electrocardiogram measuring device according to claim 6, wherein a second generating unit connector to which chair connecting electric wires are connected is attached to the electrocardiogram generating unit to connect the chair connecting electric wires connected to the electrocardiogram generating unit to the chair pad part connector.

8. The electrocardiogram measuring device according to claim 6, wherein the limb measuring electrodes of the armrest parts have surfaces on which armrest part conductive sheets are mounted, and acquire electrocardiographic signals from both palms of the subject via the armrest part conductive sheets, and the limb measuring electrodes of the footrest parts have surfaces on which footrest part conductive sheets are mounted, and acquire electrocardiographic signals from both feet soles of the subject via the footrest part conductive sheets.

9. The electrocardiogram measuring device according to claims 1, wherein the number of the attachment holes of the chest measuring electrodes opened in the garment is one or more.
